(19) United States
(12) Patent Application Publication
(10) Pub. No.: US 2011/0137088 A1
(43) Pub. Date: Jun. 9, 2011

(54) Methods and Apparatuses for Producing Renewable Materials From Inhibiting Compounds

(75) Inventor: Jacob Borden, Chicago, IL (US)

(73) Assignee: BP Corporation North America Inc., Warrenville, IL (US)

(21) Appl. No.: 12/630,577

(22) Filed: Dec. 3, 2009

(51) Int. Cl. C07C 31/02; C12P 1/00; C12P 1/04; C12P 1/02; C12P 7/02; C12P 7/06; C12P 7/16; C12P 7/64; C12P 7/62; C12P 5/02; C12P 7/56; C12P 7/52; C12P 7/54; C12M 1/00

(52) U.S. Cl. 568/840; 435/41; 435/170; 435/171; 435/155; 435/161; 435/160; 435/134; 435/135; 435/167; 435/139; 435/141; 435/140; 435/289.1

(57) ABSTRACT

Renewable materials made from inhibiting compounds. A method includes the step of consuming a fermentation inhibiting compound with a biological organism, and the step of producing a renewable material with the biological organism from at least a portion of the fermentation inhibiting compound. The methods may include a net balance of cofactor production and consumption.

### BACKGROUND

1. Technical Field

This invention relates to methods and apparatuses for producing renewable materials from inhibiting compounds. The invention also relates to renewable materials made by the methods and apparatuses of this invention.

2. Discussion of Related Art

Issues of greenhouse gas levels and climate change have led to development of technologies seeking to utilize natural cycles between fixed carbon and liberated carbon dioxide. As these technologies advance, various techniques to convert feedstocks into renewable materials have been developed. However, even with the above advances in technology, there remains a need and a desire to increase yield of the renewable material and extend a range of compounds that can be converted into the renewable material. A further complication to the conversion of a broader range of compounds is cofactor imbalances that can lead to sub-optimal yields. There is also a need and a desire for methods and/or processes with a net balance of cofactor production and consumption.

### SUMMARY

This invention relates to methods and apparatuses for producing renewable materials from inhibiting compounds. The invention also relates to renewable materials made by the methods and apparatuses of this invention. This invention can utilize and/or consume materials and/or compounds that would otherwise inhibit and/or reduce output of renewable materials and convert and/or transform the materials and/or compounds into additional renewable materials. Benefits of the invention may include increased yield of the renewable material, reduced waste, lowered feedstock costs, and/or the like. This invention may also include methods and/or processes with a net balance of cofactor production and consumption.

According to a first embodiment, this invention includes a method of producing renewable materials. The method includes the step of consuming an inhibiting compound with a biological organism, and the step of producing a renewable material with the biological organism from at least a portion of the inhibiting compound.

According to a second embodiment, this invention includes a method of producing renewable materials. The method includes the step of consuming two aldose molecules with a biological organism to produce two polyol molecules, and the step of consuming the two polyol molecules with the biological organism to produce two ketose molecules and two reduced cofactor molecules. The method includes the step of consuming the two ketose molecules with the biological organism to produce a renewable material molecule, and the step of consuming an organic acid molecule and one reduced cofactor molecule with the biological organism to produce an aldehyde molecule and an oxidized cofactor molecule. The method includes the step of consuming the aldehyde molecule and one reduced cofactor molecule with the biological organism to produce a same or a different renewable material molecule than formed from the two ketose molecules and an oxidized cofactor molecule.

According to a third embodiment, the invention includes an apparatus for producing renewable materials. The apparatus includes a fermentation vessel adapted to receive a lignocellulosic stream, and a biological organism disposed within the fermentation vessel. The biological organism includes genes of xylose reductase, xylitol dehydrogenase, coenzyme A transferase, acetaldehyde dehydrogenase, and/or alcohol dehydrogenase.

According to a fourth embodiment, the invention includes a renewable material made by any of the methods and/or apparatuses disclosed within this specification.

### BRIEF DESCRIPTION OF THE DRAWING

The accompanying drawings, which are incorporated in and constitute a part of this specification, illustrate embodiments of the invention and, together with the description, serve to explain the features, advantages, and principles of the invention. In the drawings:

FIG. 1 shows a pathway from acetic acid to ethanol, according to one embodiment;

FIG. 2 shows a pathway from butyric acetic acid to butanol, according to one embodiment;

FIG. 3 shows net pathways from acetic acid to ethanol and xylose to ethanol, according to one embodiment; and

FIG. 4 shows an apparatus, according to one embodiment.

### DETAILED DESCRIPTION

The invention relates to methods and apparatuses for producing renewable materials from inhibiting compounds. The invention also relates to renewable materials made by the methods and apparatuses disclosed within this specification. According to one embodiment, the invention may include conversion of fermentation inhibitors into valuable products and/or coproducts.

Pretreatment of lignocellulosic biomass can yield carbohydrates for conversion to renewable fuels. Pretreatment can also result in solvation and/or synthesis of organic acids, such as from side-chain acyl substitutions in hemicellulose, natural degradation of hexose, pentose, or other carbohydrate monomers, and/or the like. Organic acids formed during pretreatment can inhibit and/or potentially preclude microbial growth on lignocellulosic carbohydrates, resulting in decreased conversion of liberated sugars to renewable material molecules and/or biofuel molecules. Mitigation of the impact of these organic acids may include use of costly sorbents and/or water-intensive wash steps for acid removal.

According to one embodiment, several microbes, for example Clostridium acetobutylicum, can contain metabolic pathways for the assimilation of organic acids and their subsequent conversion to valuable biofuel molecules, such as acetic acid to ethanol, butyric acid to butanol, and/or the like.

The invention may include a use of organisms in production of renewable materials and/or biofuels, such as Saccharomyces cerevisiae 424A (LNH-ST), Escherichia coli KO11, Zymomonas mobilis AX101, and/or the like. According to one embodiment, the invention also may include use of genes to catalyze organic acid assimilation reactions and/or biofuel synthesis reactions including anabolic and/or catabolic pathways. Strains of organisms can be genetically modified to incorporate the organic acid assimilation reactions and/or the biofuel synthesis genes. The genetic modifications may include control of a promoter with a temporal and/or inducible expression pattern, and/or the like. For example, a suitable genetic modification may include an expression pattern during an early growth phase. Acid assimilation genes can be turned on during the early stages of growth allowing for detoxification of pretreated biomass while simultaneously generating valuable renewable material molecules and/or biofuel molecules from originally inhibitory compounds and/or organic acids.

According to one embodiment, the invention may include the use of inhibitory compounds formed and/or solvated during the pretreatment of lignocellulosic biomass, such inhibitory compounds may include organic acids that can inhibit microbial growth, limit the conversion of lignocellulosic derived sugars to renewable materials and/or biofuels, and/or the like. Utilizing metabolic pathways that convert organic acids into alcohols can mitigate the above issues. Genes forming metabolic pathways can be used to genetically modify organisms that naturally produce renewable materials and/or biofuels to convert toxic organic acids into valuable renewable material molecules and/or biofuel molecules.

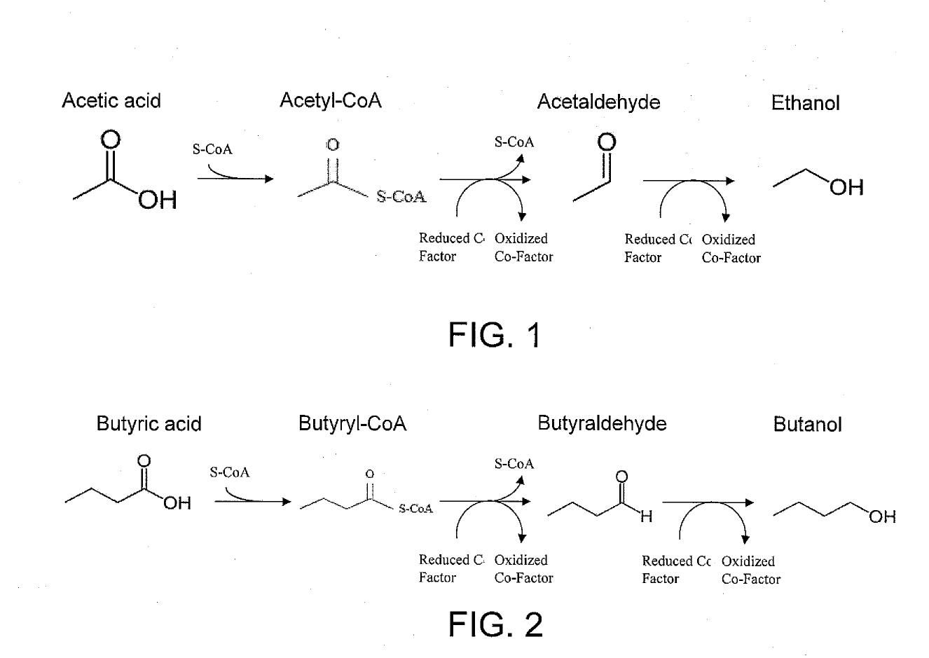

FIG. 1 shows a pathway from acetic acid to ethanol, according to one embodiment.

FIG. 2 shows a pathway from butyric acetic acid to butanol according to one embodiment.

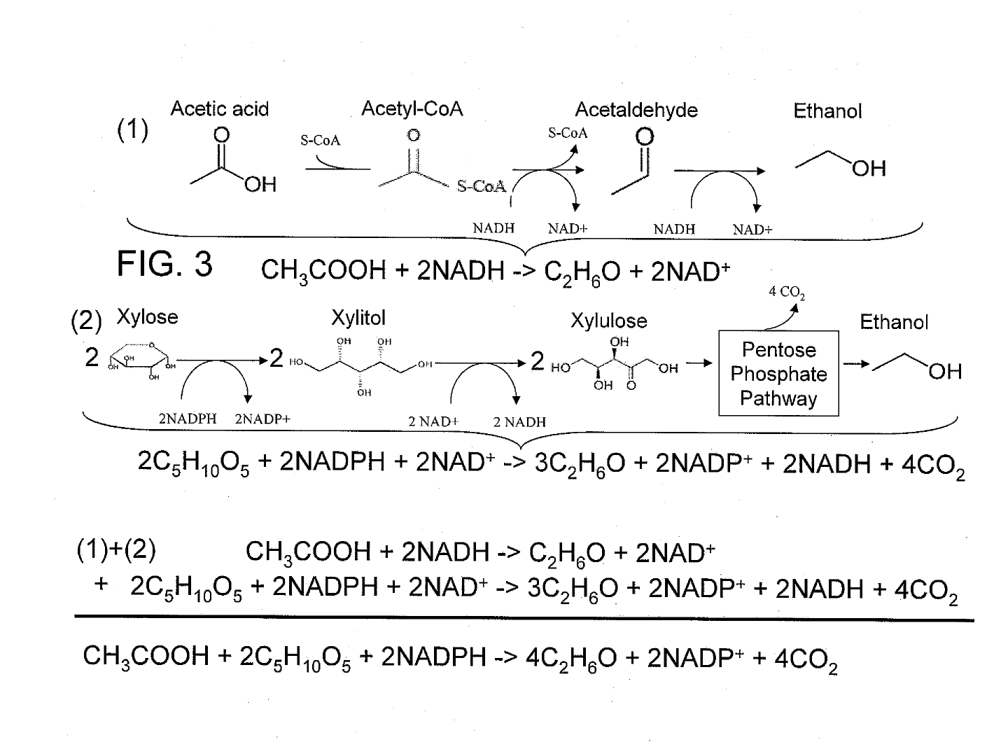

FIG. 3 shows pathways from acetic acid to ethanol (1) and xylose to ethanol (2), according to one embodiment. The bottom of the figure shows the net chemical reaction (1+2) including a net zero (balanced) production and consumption of cofactors.

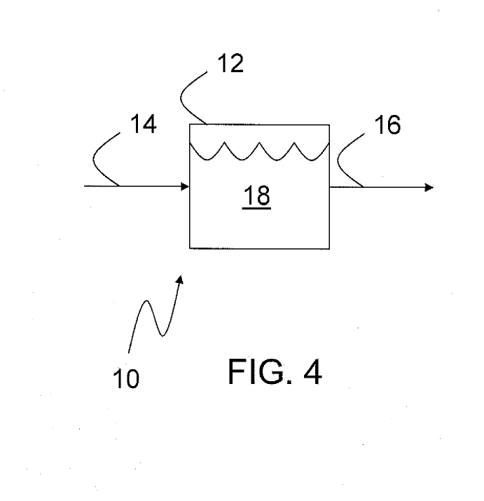

FIG. 4 shows an apparatus 10, according to one embodiment. The apparatus 10 includes a vessel 12 with a lignocellulosic stream (feedstock) 14 and a renewable material stream (product) 16. The vessel 12 includes a biological organism 18 disposed within the vessel 12.

According to one embodiment, the invention may include a method of producing renewable materials. The method may include the step of consuming a fermentation inhibiting compound with a biological organism, and the step of producing a renewable material with the biological organism from at least a portion of the fermentation inhibiting compound.

Method broadly refers to a procedure and/or a process for attaining and/or achieving an outcome and/or a result.

According to one embodiment, the steps of the method may exclude a water wash step to remove organic acids and/or other inhibiting compounds. In the alternative, the steps of the method may include a reduced water wash step.

Renewable materials broadly refer to substances and/or items that have been at least partially derived from a source and/or process capable of being replaced by natural ecological cycles and/or resources. Renewable materials may broadly include chemicals, chemical intermediates, solvents, monomers, oligomers, polymers, biofuels, biofuel intermediates, biogasoline, biogasoline blendstocks, biodiesel, green diesel, renewable diesel, biodiesel blend stocks, biodistillates, and/or the like. Desirably, but not necessarily, the renewable material may be derived from a living organism, such as plants, algae, bacteria, fungi, and/or the like.

Biofuel broadly refers to components and/or streams suitable for use as a fuel and/or a combustion source derived from renewable sources. The biofuel may be sustainably produced and/or have reduced and/or no net carbon emissions to the atmosphere. According to one embodiment, renewable sources may exclude materials mined or drilled, such as from the underground. Desirably, renewable resources may include single cell organisms, multicell organisms, plants, fungi, bacteria, algae, cultivated crops, non-cultivated crops, timber, and/or the like. Biofuels may be suitable for use as transportation fuels, such as for use in land vehicles, marine vehicles, aviation vehicles, and/or the like. Biofuels may be suitable for use in power generation, such as raising steam, generating syngas, generating hydrogen, making electricity, and or the like.

Biogasoline broadly refers to components and/or streams suitable for direct use and/or blending into a gasoline pool and/or an octane supply derived from renewable sources. Suitable biogasoline molecules may include methane, hydrogen, syngas (synthesis), methanol, ethanol, propanol, butanol, dimethyl ether, methyl tert-butyl ether, ethyl tert-butyl ether, hexanol, aliphatic compounds (straight, branched, and/or cyclic), heptane, isooctane, cyclopentane, aromatic compounds, ethyl benzene, and/or the like. Butanol broadly refers to products and derivatives of 1-butanol, 2-butanol, iso-butanol, other isomers, and/or the like. Biogasoline may be used in spark-ignition engines, such as automobile gasoline internal combustion engines. According to one embodiment, the biogasoline and/or biogasoline blends meet or comply with industrially accepted fuel standards.

Biodiesel broadly refers to components or streams suitable for direct use and/or blending into a diesel pool and/or a cetane supply derived from renewable sources. Suitable biodiesel molecules may include fatty acid esters, triglycerides, lipids, fatty alcohols, alkanes, naphthas, distillate range materials, paraffinic materials, aromatic materials, aliphatic compounds (straight, branched, and/or cyclic), and/or the like. Biodiesel may be used in compression-ignition engines, such as automotive diesel internal combustion engines. In the alternative, the biodiesel may also be used in gas turbines, heaters, boilers, and/or the like. According to one embodiment, the biodiesel and/or biodiesel blends meet or comply with industrially accepted fuel standards.

Biodistillate broadly refers to components or streams suitable for direct use and/or blending into aviation fuels (jet), lubricant base stocks, kerosene fuels, and/or the like. Biodistillate can be derived from renewable sources, and have a boiling point range of between about 100 degrees Celsius and about 700 degrees Celsius, between about 150 degrees Celsius and about 350 degrees Celsius, and/or the like.

Biomass broadly refers to plant and/or animal materials and/or substances derived at least in part from living organisms and/or recently living organisms, such as lignocellulosic sources.

Lignocellulosic broadly refers to containing cellulose, hemicellulose, lignin, and/or the like. Lignocellulosic may refer to plant material. Lignocellulosic material may include any suitable material, such as sugar cane, sugar cane bagasse, energy cane, energy cane bagasse, rice, rice straw, corn, corn stover, wheat, wheat straw, maize, maize stover, sorghum, sorghum stover, sweet sorghum, sweet sorghum stover, cotton, cotton remnant, sugar beet, sugar beet pulp, soybean, rapeseed, jatropha, switchgrass, miscanthus, other grasses, timber, softwood, hardwood, wood bark, wood waste, sawdust, paper, paper waste, agricultural waste, manure, dung, sewage, municipal solid waste, any other suitable biomass material, and/or the like.

Consuming broadly refers to use up, utilize, eat, devour, transform, and/or the like, such as during cellular metabolism, cellular respiration (aerobic and/or anaerobic), cellular reproduction, cellular growth, fermentation, cell culturing, and/or the like.

Fermentation broadly refers to the metabolism of carbohydrates where a final electron donor is not oxygen, such as anaerobically. Fermentation may include an enzyme controlled anaerobic breakdown of an energy-rich compound, such as a carbohydrate to carbon dioxide and an alcohol and/or an organic acid. In the alternative, fermentation broadly refers to biologically-controlled transformation of an organic compound. Fermentation processes may use any suitable organisms, such as bacteria, fungi, algae, and/or the like. Suitable fermentation processes may include naturally occurring organisms and/or genetically modified organisms.

Enzymes broadly refer to proteins or other suitable molecules to catalyze and/or increase chemical reactions, biochemical reactions, and/or the like. Enzymes may be produced by living organisms and/or synthetic processes. Suitable enzymes may include any desirable property and/or characteristic. Suitable enzymes may include cellulase, hemicellulase, ligninase, endo-cellulase, exo-cellulase, glucosidase, cellobiose dehydrogenase, manganese peroxidase, lignin peroxidase, and/or the like. Suitable enzymes may aid hydrolysis of cellulose to smaller sugar units and/or monomers.

Genetic broadly refers to heredity and/or variety of organisms, such as expressed through series and/or sequences of nucleic acid in polymer chain forms, on chromosomes, and/or the like.

Nucleic acid broadly refers to complex organic acids of nucleotide chains, such as deoxyribonucleic acid (DNA), ribonucleic acid (RNA), and/or the like.

Gene broadly refers to a specific sequence of nucleotides in DNA and/or RNA as a functional unit of inheritance for controlling a transmission and/or an expression of one or more traits and/or attributes. Genes may specify a structure of a polypeptide and/or a protein, control a function of other genetic material, and/or the like.

Genetic engineering broadly refers to intentional manipulation and/or modification of at least a portion of a genetic code and/or expression of a genetic code of an organism.

Genetically modified broadly refers to organisms, cultures, single-cells, biota, and/or the like that have been genetically engineered. Genetically modified organisms may include those manipulated by genomic mutagenesis, addition and/or removal of one or more genes, portions of proteins, promoter regions, non-coding regions, chromosomes, and/or the like.

Naturally occurring broadly refers to organisms, cultures, single-cells, biota, and/or the like at least generally without intervening actions by exterior forces, such as humankind, machine, and/or the like. Naturally occurring organisms may include those found in local environments (flora and/or fauna) and/or the like. Naturally occurring organisms may be collected, isolated, cultured, purified, and/or the like.

Cell culturing broadly refers to the metabolism of carbohydrates whereby a final electron donor is oxygen, such as aerobically. Cell culturing processes may use any suitable organisms, such as bacteria, fungi, algae, and/or the like. Suitable cell culturing processes may include naturally occurring organisms and/or genetically modified organisms.

Inhibit broadly refers to hold in check, restrain, impede production, curtail growth, slow a process, and/or the like.

Compound broadly refers to any suitable element and/or material, combination of elements, and/or the like.

Inhibiting compounds broadly refer to elements, substances, and/or compounds that slow cellular operations and/or impede production of desired products. Inhibiting compounds may broadly include alcohols, organic acids, and/or the like. Fermentation inhibiting compounds may include any suitable substance and/or material, such as formic acid, acetic acid, butyric acid, lactic acid, pyruvic acid, propionic acid, furfural, hydroxymethylfurfural, phenymethylethers, other pretreatment byproducts, other fermentation byproducts, and/or the like. Without being bound by theory, some inhibiting compounds can shift equilibrium away form a desired product, block pathways, disrupt cellular processes, alter genetic cascades, and/or the like. Some inhibiting compounds may have an inhibiting amount upon, such as upon reaching a threshold concentration.

Inhibiting compounds may slow and/or retard cellular activities by any suitable amount, such as by at least about 1 percent, at least about 5 percent, at least about 10 percent, at least about 30 percent, at least about 70 percent, and/or the like on a mass and/or volume output basis versus a system without the inhibiting compound.

The inhibiting compounds may come from and/or be derived from any suitable source, such as fermentation of sugars (byproducts), hydrolysis of lignocellulosic material, and/or the like.

Biological broadly refers to life systems, living processes, alive organisms, and/or the like. Biological may refer to organisms from archaea, bacteria, and/or eukarya. According to one embodiment, biological includes biologically-derived compounds, such as enzymes, proteins, and/or the like. According to one embodiment, biological excludes fossilized and/or ancient materials, such as those whose life ended at least about 1,000 years ago.

Biological processes may include any suitable living system and/or item derived from a living system and/or steps. Biological processes may include fermentation, cell culturing, aerobic respiration, anaerobic respiration, catabolic reactions, anabolic reactions, biotransformation, saccharification, liquefaction, hydrolysis, depolymerization, polymerization, and/or the like.

Organism broadly refers to a complex structure of interdependent and subordinate elements whose relations and/or properties may be largely determined by their function in the whole. The organism may include an individual designed to carry on the activities of life with organs separate in function but mutually dependent. Organisms may include a living being, such as capable of growth, reproduction, and/or the like.

The biological organism may include any suitable simple (mono) cell being, complex (multi) cell being, and/or the like. Biological organisms may include algae, fungi, bacteria, and/or the like. The biological organism may include one or more naturally occurring organisms, one or more genetically modified organisms, combinations of naturally occurring organisms and generically modified organisms, and/or the like. Embodiments with combinations of multiple biological organisms are within the scope of the invention. Any suitable combination or organism can be used, such as 1 or more organisms, at least about 2 organisms, at least about 5 organisms, between about 2 organisms and about 20 organisms, and/or the like.

The biological organism may include any suitable organism, such as Aspergillus nidulans, Bacillus subtilis, Candida curvata, Candida sorensis, Clostridium acetobutylicum, Clostridium beijerinckii, Crypthecodinium cohnii, Cryptococcus terricolus, Entomorphtoria coronata, Escherichia coli, Geobacter thermoglucosidasius, Hansenula polymorpha, Klebsiella oxytoca, Kluyveromyces marxianus, Lipomyces lipfer, Moorella thermoaceticum, Mortierella alpine, Pichia stipitis, Rhodosporidium toruloides, Rhodotorula glutinis, Saccharomyces bayanus, Saccharomyces carlsbergensis, Saccharomyces cerevisiae, Saccharomyces pastorianus, Schyzochytrium spp., Trichoderma Reesei, Trichosporon cutaneum, Yarrowia lipolytica, Zymomonas mobilis, and/or the like.

Produce broadly refers to bring into existence, to create, to make, to synthesize, and/or the like. According to one embodiment, producing includes fermentation, cell culturing, and/or the like. Producing may include making renewable materials within an organism. Processes may include further processing to extract and/or remove the removable material by cell lysing, mechanical processing, thermal processing, chemical processing, and/or the like. In the alternative, the producing may excrete and/or discharge the renewable material from the organism without additional steps and/or processing.

Portion broadly refers to a part, a share, a piece, a component of a whole, and/or the like.

According to one embodiment, the method can also include consumption of a sugar material by a biological organism to produce at least a portion of the inhibiting compound. Sugars broadly refer to any suitable carbohydrate, such as monosaccharides, disaccharides, trisaccharides, and/or the like. Sugars may include any suitable number of carbon atoms, such as about 5 (pentose), about 6 (hexose), and/or the like.

The method may include any suitable production and/or consumption of cofactors. The method may be a net consumer of cofactors, a net producer of cofactors, a balanced producer and consumer of cofactors, and/or the like. Desirably, but not necessarily, the method may include a net consumption and/or production of cofactors equal to about 0. The cofactors may include redox-potential cofactor NADH, NADPH, and/or the like. Without being bound by theory, production of renewable materials can benefit from methods and processes having a balanced requirement for production and/or consumption of cofactors. Balanced cofactor systems can provide at least somewhat self-sustaining production of renewable materials.

Net broadly refers to remaining after accounting for all gains and/or losses. Net may refer to being free from additional charges, and/or the like.

NADH broadly refers to a reduced state and/or form of nicotinamide adenine dinucleotide which can be a coenzyme.

NADPH broadly refers to a reduced state and/or form of nicotinamide adenine dinucleotide phosphate which can be a coenzyme.

According to one embodiment, the biological organism may have been genetically modified to convert the fermentation inhibiting compound to an alcohol through an aldehyde pathway with coenzyme A and two units of a reduced cofactor, such as NADH.

Alcohol broadly refers to a class of compounds containing a hydroxyl group (OH⁻), such as ethanol, propanol, butanol, and/or the like.

Aldehyde broadly refers to a class of compounds containing a carbonyl group (O═CH⁻), such as acetaldehyde, formaldehyde, and/or the like.

Pathway broadly refers to a sequence and/or an order of steps and/or reactions. Suitably pathways may include enzyme-catalyzed reactions by which one substance can be converted into another, and/or the like.

Coenzyme broadly refers to a thermostable nonprotein compound. The coenzyme can form an active portion of an enzyme system after combination with an apoenzyme, and/or the like.

Apoenzyme broadly refers to a protein that forms an active enzyme system by combination with a coenzyme and determines a specificity of a system for a substrate, and/or the like.

Coenzyme A broadly refers to coenzyme with a formula of C₂₁H₃₆N₇O₁₆P₃S. Without being bound by theory, coenzyme A can be used in metabolism of carbohydrates, fats, amino acids, and/or the like. Coenzyme A can be used in synthesis of fatty acids, such as for use in renewable materials.

The biological organism may use any suitable enzymes and/or genes for production of the renewable material. Suitable genes may include alcohol dehydrogenase, aldehyde dehydrogenase, aldehyde reductase, and/or the like.

According to one embodiment, the biological organism uses one or more alcohol dehydrogenase units. Alcohol dehydrogenase broadly refers to a group of enzymes for conversion to and/or from alcohols and other compounds.

Unit broadly refers to a single quantity regarded as a whole, an amount of a biologically active agent to produce a result, and/or the like.

According to one embodiment, the biological organism uses one or more aldehyde dehydrogenase units. Aldehyde dehydrogenase broadly refers to a group of enzymes for catalyzing oxidation of aldehydes.

According to one embodiment, the biological organism uses one or more aldehyde reductase units. Aldehyde reductase broadly refers to a group of enzymes for used for carbohydrate metabolism, such as conversion of glucose to sorbitol.

According to one embodiment, the biological organism uses one or more units of coenzyme A transferase, one or more units of acetaldehyde dehydrogenase, and/or one or more units of alcohol dehydrogenase.

According to one embodiment, the renewable material may include ethanol, butanol, free fatty acids, triacylglycerides, alkyl esters, isoprenoids, lactic acid, acetic acid, butyric acid, propionic acid, other hydrocarbons, and/or the like.

According to one embodiment, the step of consuming and the step of producing occur independent of sugar fermentation, such as in separate vessel. In the alternative, the step of consuming and the step of producing occur at least somewhat contemporaneous and/or simultaneously with sugar fermentation, such as in a same vessel.

Embodiments with simultaneous conversion of sugars to renewable materials during consumption of inhibiting compounds are within the scope of, this invention. The same and/or different organisms may be used to consume the sugar and the inhibiting compounds.

According to one embodiment, the invention may include a renewable material made by any of the methods, and/or apparatuses described in this specification.

According to one embodiment, the invention may include a second method of producing renewable materials. The method may include the step of consuming at least two aldose molecules with a biological organism to produce at least two polyol molecules, and the step of consuming the at least two polyol molecules with the biological organism to produce at least two ketose molecules and at least two reduced cofactor molecules. The method may include the step of consuming the at least two ketose molecules with the same biological organism and/or a different biological organism to produce at least one renewable material molecule, and the step of consuming an organic acid molecule and one reduced cofactor molecule with the biological organism to produce at least an aldehyde molecule and at least an oxidized cofactor molecule. The method may include the step of consuming at least the aldehyde molecule and at least one reduced cofactor molecule with the same biological organism and/or a different biological organism to produce a same and/or a different renewable material molecule than formed from the at least two ketose molecules and at least an oxidized cofactor molecule.

The second method may include any of the steps, features, and/or characteristics of the first methods, the products, and/or the apparatuses described in this specification and vice verse.

Molecule broadly refers to a smallest particle and/or portion of a substance and/or material that retains all the properties of the substance and/or material. Molecules may include of one or more atoms.

Aldose broadly refers to a monosaccharide (simple sugar) with one aldehyde group per molecule. Aldose may have a chemical formula of Cₙ(H₂O)ₙ. Aldose molecules may include xylose, and/or the like.

Polyol broadly refers to alcohols with multiple hydroxyl groups which may be sometimes be referred to as a sugar alcohol. Polyols may include xylitol, sorbitol, and/or the like.

Ketose broadly refers to molecules with one ketone group. Ketoses may sometimes be referred to as reducing sugars. Ketose molecules may include xylulose, and/or the like.

Reduced broadly refers to having added one or more electrons to an atom, an ion, an element, a molecule, and/or a compound, to having changed an atom, an ion, an element, a molecule, and/or a compound from a higher to a lower oxidation state, to having combined with hydrogen, to having been subjected to an action of hydrogen, and/or like.

Organic broadly refers to carbon-containing compounds, such as carbohydrates, sugars, ketones, aldehydes, alcohols, lignin, cellulose, hemicellulose, pectin, other carbon containing substances, and/or the like.

Acid broadly refers to materials and/or substances with a pH less than about 7, proton donors, electron acceptors, and/or the like. Acids may include organic acids, mineral acids, and/or the like.

Organic acid broadly refers acid materials and/or substances containing one or more carbon atoms. Organic acids may include formic acid, acetic acid, lactic acid, butyric acid, other carboxylic acids, and/or the like.

Oxidized broadly refers to having changed an atom, an ion, an element, a molecule, and/or a compound by increasing an electronegative part and/or change from a lower to a higher positive valence, to having removed one or more electrons from an atom, an ion, an element, a molecule, and/or a compound, and/or the like.

According to one embodiment of the second method, the organic acid molecule may include acetate and/or butyrate, and/or the like, and the aldehyde molecule may include acetaldehyde, butyraldehyde, and/or the like.

The biological organism of the second method may include any suitable living being and/or derivative thereof, such as including archaea, bacteria, eukaryotes and/or the like. Embodiments with combinations of multiple biological organisms are within the scope of the invention.

According to one embodiment, the reduced cofactor may include NADH and/or the like.

The biological organism of the second method may include any suitable organism, such as Aspergillus nidulans, Bacillus subtilis, Candida curvata, Candida sorensis, Clostridium acetobutylicum, Clostridium beijerinckii, Crypthecodinium cohnii, Cryptococcus terricolus, Entomorphtoria coronata, Escherichia coli, Geobacter thermoglucosidasius, Hansenula polymorpha, Klebsiella oxytoca, Kluyveromyces marxianus, Lipomyces lipfer, Moorella thermoaceticum, Mortierella alpine, Pichia stipitis, Rhodosporidium toruloides, Rhodotorula glutinis, Saccharomyces bayanus, Saccharomyces carlsbergensis, Saccharomyces cerevisiae, Saccharomyces pastorianus, Schyzochytrium spp., Trichoderma Reesei, Trichosporon cutaneum, Yarrowia lipolytica, Zymomonas mobilis, and/or the like

According to one embodiment of the second method, the biological organism may include genes of one or more xylose reductases, one or more xylitol dehydrogenases, and/or one or more alcohol dehydrogenases.

According to one embodiment of the second method, the biological organism may include genes of one or more xylose reductases, xylitol dehydrogenases, and/or one or more aldehyde dehydrogenases.

According to one embodiment of the second method, the biological organism may include genes of one or more xylose reductases, one or more xylitol dehydrogenases, and/or one or more aldehyde reductases.

According to one embodiment of the second method, the biological organism may include genes of one or more xylose reductases, one or more xylitol dehydrogenases, one or more coenzyme A transferases, one or more acetaldehyde dehydrogenases, and/or one or more alcohol dehydrogenases.

The renewable material of the second method may include any suitable material, such as a biofuel, a chemical, and/or the like. Biofuels may include ethanol, butanol, free fatty acids, triacylglycerides, alkyl esters, isoprenoids, lactic acid, acetic acid, butyric acid, propionic acid, and/or the like.

According to one embodiment of the second method, a net consumption and production of redox-potential cofactor NADH equals about 0.

According to one embodiment, the invention may include a renewable material made by any of the second methods described in this specification.

According to one embodiment, the invention may include an apparatus for producing renewable materials. The apparatus may include a fermentation vessel adapted to receive a lignocellulosic stream, and a biological organism disposed within the fermentation vessel. The biological organism may be genetically modified to include genes of xylose reductase, xylitol dehydrogenase, coenzyme A transferase, acetaldehyde dehydrogenase, and/or alcohol dehydrogenase.

Apparatus broadly refers to a set of materials, devices, and/or equipment designed for one or more particular use, purpose, outcome, and/or the like.

Vessel broadly refers to a container and/or holder of a substance, such as a liquid, a gas, a fermentation broth, and/or the like. Vessels may include any suitable size and/or shape, such as at least about 1 liter, at least about 1,000 liters, at least about 100,000 liters, at least about 1,000,000 liters, and/or the like. Vessels may include any suitable auxiliary equipment, such as pumps, agitators, heat exchangers, coils, pressurization systems (vacuum), control systems, and/or the like.

Stream broadly refers to a flow and/or a supply of a substance and/or a material, such as a steady succession. Flow of streams may be continuous, discrete, intermittent, batch, semi-batch, semi-continuous, and/or the like.

Dispose broadly refers to put in place, to put in location, to set in readiness, and/or the like.

Adapted broadly refers to make fit for a specific use, purpose, and/or the like.

As used herein the terms “has”, “having”, “comprising” “with”, “containing”, and “including” are open and inclusive expressions. Alternately, the term “consisting” is a closed and exclusive expression. Should any ambiguity exist in construing any term in the claims or the specification, the intent of the drafter is toward open and inclusive expressions.

As used herein the term “and/or the like” provides support for any and all individual and combinations of items and/or members in a list, as well as support for equivalents of individual and combinations of items and/or members.

Regarding an order, number, sequence, and/or limit of repetition for steps in a method or process, the drafter intends no implied order, number, sequence and/or limit of repetition for the steps to the scope of the invention, unless explicitly provided.

Regarding ranges, ranges are to be construed as including all points between upper values and lower values, such as to provide support for all possible ranges contained between the upper values and the lower values including ranges with no upper bound and/or lower bound.

It will be apparent to those skilled in the art that various modifications and variations can be made in the disclosed structures and methods without departing from the scope or spirit of the invention. Particularly, descriptions of any one embodiment can be freely combined with descriptions of other embodiments to result in combinations and/or variations of two or more elements and/or limitations. Other embodiments of the invention will be apparent to those skilled in the art from consideration of the specification and practice of the invention disclosed herein. It is intended that the specification and examples be considered exemplary only, with a true scope and spirit of the invention being indicated by the following claims.

What is claimed is:

1. A method of producing renewable materials, the method comprising:
consuming a fermentation inhibiting compound with a biological organism; and
producing a renewable material with the biological organism from at least a portion of the fermentation inhibiting compound.

2. The method of claim 1, wherein a net consumption and production of redox-potential cofactor NADH equals about 0.

3. The method of claim 1, wherein the fermentation inhibiting compound comprises an organic acid.

4. The method of claim 1, wherein the fermentation inhibiting compound comprises formic acid, acetic acid, butyric acid, lactic acid, pyruvic acid, propionic acid, furfural, hydroxymethylfurfural, phenymethylethers, or combinations thereof.

5. The method of claim 1, wherein the biological organism comprises archaea, bacteria, eukaryotes or combinations thereof.

6. The method of claim 1, wherein the biological organism comprises Aspergillus nidulans, Bacillus subtilis, Candida curvata, Candida sorensis, Clostridium acetobutylicum, Clostridium beijerinckii, Crypthecodinium cohnii, Cryptococcus terricolus, Entomorphtoria coronata, Escherichia coli, Geobacter thermoglucosidasius, Hansenula polymorpha, Klebsiella oxytoca, Kluyveromyces marxianus, Lipomyces lipfer, Moorella thermoaceticum, Mortierella alpine, Pichia stipitis, Rhodosporidium toruloides, Rhodotorula glutinis, Saccharomyces bayanus, Saccharomyces carlsbergensis, Saccharomyces cerevisiae, Saccharomyces pastorianus, Schyzochytrium spp., Trichoderma Reesei, Trichosporon cutaneum, Yarrowia lipolytica, Zymomonas mobilis, or combinations thereof.

7. The method of claim 1, wherein the biological organism has been genetically modified to convert the fermentation inhibiting compound to an alcohol through an aldehyde pathway with coenzyme A and two units of a reduced cofactor.

8. The method of claim 7, wherein the reduced cofactor is NADH.

9. The method of claim 1, wherein the biological organism uses one or more alcohol dehydrogenase units.

10. The method of claim 1, wherein the biological organism uses one or more aldehyde dehydrogenase units.

11. The method of claim 1, wherein the biological organism uses one or more aldehyde reductase units.

12. The method of claim 1, wherein the biological organism uses coenzyme A transferase, acetaldehyde dehydrogenase, and alcohol dehydrogenase.

13. The method of claim 1, wherein the renewable material comprises a biofuel.

14. The method of claim 1, wherein the renewable material comprises ethanol, butanol, free fatty acids, triacylglycerides, alkyl esters, isoprenoids, lactic acid, acetic acid, butyric acid, propionic acid, or combinations thereof.

15. The method of claim 1, wherein the step of consuming and the step of producing occur independent of sugar fermentation.

16. The method of claim 1, wherein the step of consuming and the step of producing occur simultaneously with sugar fermentation.

17. The method of claim 1, wherein the fermentation inhibiting compound comes from hydrolysis of lignocellulosic material.

18. A renewable material made by the method of claim 1.

19. A method of producing renewable materials, the method comprising:
consuming two aldose molecules with a biological organism to produce two polyol molecules;
consuming the two polyol molecules with the biological organism to produce two ketose molecules and two reduced cofactor molecules;
consuming the two ketose molecules with the biological organism to produce a renewable material molecule;
consuming an organic acid molecule and one reduced cofactor molecule with the biological organism to produce an aldehyde molecule and an oxidized cofactor molecule; and
consuming the aldehyde molecule and one reduced cofactor molecule with the biological organism to produce a same or a different renewable material molecule than formed from the two ketose molecules and an oxidized cofactor molecule.

20. The method of claim 19, wherein:
the aldose molecules comprise xylose;
the polyol molecules comprise xylitol; and
the ketose molecules comprise xylulose

21. The method of claim 19, wherein:
the organic acid molecule comprises acetate or butyrate; and
the aldehyde molecule comprises acetaldehyde or butyraldehyde.

22. The method of claim 19, wherein the biological organism comprises archaea, bacteria, eukaryotes or combinations thereof.

23. The method of claim 19, wherein the reduced cofactor is NADH.

24. The method of claim 19, wherein the biological organism comprises Aspergillus nidulans, Bacillus subtilis, Candida curvata, Candida sorensis, Clostridium acetobutylicum, Clostridium beijerinckii, Crypthecodinium cohnii, Cryptococcus terricolus, Entomorphtoria coronata, Escherichia coli, Geobacter thermoglucosidasius, Hansenula polymorpha, Klebsiella oxytoca, Kluyveromyces marxianus, Lipomyces lipfer, Moorella thermoaceticum, Mortierella alpine, Pichia stipitis, Rhodosporidium toruloides, Rhodotorula glutinis, Saccharomyces bayanus, Saccharomyces carlsbergensis, Saccharomyces cerevisiae, Saccharomyces pastorianus, Schyzochytrium spp., Trichoderma Reesei, Trichosporon cutaneum, Yarrowia lipolytica, Zymomonas mobilis, or combinations thereof.

25. The method of claim 19, wherein the biological organism comprises genes of xylose reductase, xylitol dehydrogenase, and one or more alcohol dehydrogenase.

26. The method of claim 19, wherein the biological organism comprises genes of xylose reductase, xylitol dehydrogenase, and one or more aldehyde dehydrogenase.

27. The method of claim 19, wherein the biological organism comprises genes of xylose reductase, xylitol dehydrogenase, and one or more aldehyde reductase.

28. The method of claim 19, wherein the biological organism comprises genes of xylose reductase, xylitol dehydrogenase, coenzyme A transferase, acetaldehyde dehydrogenase, and alcohol dehydrogenase.

29. The method of claim 19, wherein the renewable material comprises a biofuel.

30. The method of claim 19, wherein the renewable material molecules comprise ethanol, butanol, free fatty acids, triacylglycerides, alkyl esters, isoprenoids, lactic acid, acetic acid, butyric acid, propionic acid, or combinations thereof.

31. The method of claim 19, wherein a net consumption and production of redox-potential cofactor NADH equals about 0.

32. A renewable material made by the method of claim 19.

33. An apparatus for producing renewable materials, the apparatus comprising:
a fermentation vessel adapted to receive a lignocellulosic stream; and
a biological organism disposed within the fermentation vessel and the biological organism comprising genes of xylose reductase, xylitol dehydrogenase, coenzyme A transferase, acetaldehyde dehydrogenase, and alcohol dehydrogenase.
